(19) United States
(12) Patent Application Publication
(10) Pub. No.: US 2009/0048625 A1
(43) Pub. Date: Feb. 19, 2009

(54) Apparatus, Particularly for use in Laparoscopic Surgery

(75) Inventors: Terje S Pedersen, Sandnes (NO); Reza Hezari, Trondheim (NO)

Correspondence Address: KNOBBE MARTENS OLSON & BEAR LLP, 2040 MAIN STREET, FOURTEENTH FLOOR, IRVINE, CA, 92614, US

(73) Assignee: SurgiTech Norway AS, Trondheim (NO)

(21) Appl. No.: 11/813,083

(22) PCT Filed: Dec. 23, 2005
(86) PCT No.: PCT/NO2005/000479

§ 371 Date: Jun. 25, 2008

(30) Foreign Application Priority Data
Dec. 29, 2004 (NO) 20045705

(51) Int. Cl. A61B 17/29

(52) U.S. Cl. 606/205

(57) ABSTRACT

The present invention is constituted by an instrument (1) for use in laparoscopic surgery, including a grip (2) which is provided with an actuator (3), and which effects, through a linkage (3′, 5, 6), the manipulation of an effector (9). The effector (9) is positioned at a first end portion of a tubular element (7), the tubular element (7) being attached to the grip (2) of the instrument (1). The linkage (3′, 5, 6) causes the relative movement of the actuator (3) to be non-linear relative to the relative movement of the effector (9).

The present invention relates to an apparatus, in particular an apparatus for use in laparoscopic surgery, also called “keyhole surgery”.

The object of the invention is to provide a simple manually operated apparatus or instrument exhibiting ergonomically correct properties of use and technical solutions related to more efficient utilization of the surgeon's muscles used to operate the trigger or actuator of the instrument, which gives the surgeon improved control of the instrument.

Even though laparoscopy was carried out for the first time on a human being in 1910, it was not until 1987 that the use of laparoscopic techniques took off. Since then there has been a rapid development in areas of use and surgical procedures. However, the development of laparoscopic instruments has been minimal with respect to ergonomic improvements. Scientific measurements go to show that a surgeon expends up to ten times the amount of energy to carry out the same procedure laparoscopically compared with open surgery.

A great number of designs of laparoscopic instruments are known from the U.S. Pat. Nos. 5,480,409, 5,893,878, 5,383,888, 5,792,165, 5,976,121, 5,488,441, 5,735,873 and 5,868,784 and from WO 9724072, among other documents. Even though the known instruments vary greatly in design and function, the known instruments have in common that they are constituted by a grip including one or more movable parts, a “trigger” or actuator among other things, which can be manipulated by the user, for example the surgeon, to control a tool, a so-called effector, which is connected to a cantilever end portion of a tubular element or tool rod which is connected at its other end portion to the grip.

U.S. Pat. No. 5,792,165 discloses an instrument exhibiting great flexibility with respect to the manoeuvring of an effector which has three degrees of freedom: rotation, pivoting and clamping. In addition different effectors may be connected to and removed from the tubular body of the instrument. The instrument disclosed in U.S. Pat. No. 5,792,165 may also be provided with an integrated motor and micro processor partially controlling the actions of the effector.

U.S. Pat. No. 5,383,888 discloses an instrument exhibiting essentially the same functions as the instrument of U.S. Pat. No. 5,792,165.

U.S. Pat. No. 5,976,121 discloses a grip for manipulating an instrument in connection with endoscopy, in which a tool in the form of a pair of scissors at the end of the instrument is opened/closed by means of a lever.

There are several drawbacks associated with the prior art mentioned above.

One of the drawbacks relates to the very design of the grips of the instruments which are ergonomically unfavourable in the great majority of the above-mentioned prior art techniques, because the instruments do not provide for a volar-flexed working position and/or it is necessary to move fingers to operate the instrument, and other fingers than the first finger, the so-called index finger, will have to be used to operate the main functions of the instrument. This entails that small uncontrolled movements may easily occur in the surgeon's hand portion. These movements lead to relatively large and undesired movements of the operative end portion of the instrument. A result of this unfavourable design is that in an attempt to counteract the above-mentioned undesired movements, among other things, a surgeon expends up to ten times the amount energy to carry out the same procedure laparoscopically compared with open surgery.

Another substantial drawback related to a majority of the above-mentioned instruments is that they are technically very complex, which entails that the instruments will be expensive to manufacture. Thereby, to a very large degree, the instruments are intended to be reused several times. Even though, theoretically, instruments can be disinfected 100%, the study “The Clinical suitability of laparoscopic instrumentation. A prospective clinical study of function and hygiene” carried out by Fengler, Pahlke, Bisson and Kraas at the Department of Surgery, Krankenhaus Moabit, Lehrkrankenhaus der Humboldt Universität zu Berlin, among others, shows that after cleaning, a relatively large number of instruments contain residues of blood products, which represent a potential risk of patients being subjected to contagion. This may lead to the patient becoming seriously ill and, at worst, dying.

In connection with laparoscopic surgery the surgeon works, to a great degree, with the effector of the instrument, for example a grasper, in a near-closed position. To move the effector from an initial position, which is normally fully open, to a required working position, which is near fully closed, the surgeon has to move the actuator of the instrument a relatively long way. Likewise, when the effector is formed by a pair of scissors, for example, there is a need to have the most force on the scissors when it is in a near-closed position, since it is normally in this position that cutting is effected. In known instruments for laparoscopic surgery the relationship between the movement of the actuator, in consequence of an external force applied, and the movement of the effector is substantially constant from a fully open position to a fully closed position. This means that the user has to apply just as much force to the actuator in the normally “useless” range from the fully open position of the effector to the “range of use” or the active range of the effector, which lies to a great degree in the area 0%-approximately 50% open. This means that muscle groups that are used to manipulate the actuator are utilized relatively poorly. In addition to the above-mentioned drawbacks, it is necessary in, among other things, controlling the instruments disclosed in U.S. Pat. No. 5,735,873, U.S. Pat. Nos. 5,868,784 and 5,976,121 to use several fingers to manipulate the effector. This represents particularly great drawbacks when the instrument is used for any length of time and in difficult working positions, in which the hand itself is forced into abnormal and in part twisted working positions. This could result in the operator of the instrument, for example a surgeon, abusing his musculature, thereby tiring faster and experiencing strained arms and shoulders.

The present invention has as its object to remedy or at least reduce one or more of the drawbacks related to the prior art represented by the above-mentioned patent documents, and then in particular the drawbacks related to the muscle use for the manipulation of the actuator. At the same time it is an object to provide an instrument which both exhibits a very simple construction and in which a substantial part of the components of the instrument can be produced of for example, but not limited to, plastics materials. This leads to relatively low production costs and could thereby defend the use of the instrument as a disposable item. This, again, will eliminate the problem of contagia being transmitted due to inadequate cleaning of the instrument.

The object is achieved in accordance with the invention through the features specified in the description below and in the subsequent Claims.

In one aspect the present invention is constituted by an instrument for use in laparoscopic surgery, including a grip which is provided with an actuator which is arranged to effect, via a linkage, the manipulation of an effector placed at a first end portion of a tubular element, said tubular element extending at its second end portion into the grip of the instrument and being connected to a portion of the linkage, the linkage being formed by a scissors link, which is connected at a first end portion to the grip by means of a first rotary connection, and by a lever, which is connected at a second end portion to the grip in a fourth rotary connection, the scissors link being connected at its second end portion to a first end portion of the lever by means of a third rotary connection. Thus, the linkage which transmits movement and force from the actuator to the effector, will thus make the relationship between the rotation of the actuator about said first rotary connection and the jaw or opening angle of the effector exhibit a substantially hyperbolic curve, as the linkage causes the transmission ratio between the actuator and effector to be relatively great when the actuator is in or close to its initial position or non-activated position, while at the same time the effector is in or close to its fully open position, and the transmission ratio decreases as the actuator is rotated into the grip. This leads to the surgeon or other users getting better and better control of the effector the closer the actuator gets to its fully activated position, a position most often used in laparoscopic surgery. Correspondingly, the effect of the force applied to the actuator will increase in a non-linear and essentially hyperbolic fashion in the effector as the actuator is rotated into the grip. This involves that the force applied to the actuator is substantially in inverse proportion to the relative movement of the effector.

A laparoscopic operation may last for a relatively long time. Therefore, it is very important that the instrument has the best possible adjustment to the operator's hand, both with respect to the positioning of functional devices like the actuator, but also with respect to size. Therefore, in a preferred embodiment the instrument according to the present invention is provided with a grip which is provided with at least one adjustable portion enabling adjustment of the grip to the size of the instrument operator's hand. In one embodiment the at least one adjustable portion of the instrument is placed in a back portion of the grip.

In connection with surgery there is often the need to scorch tissues or blood vessels. In a preferred embodiment the instrument is provided with an electrical connection known in itself, for the connection of power which is supplied to the effector through wires for monopolar cutting to be carried out.

In what follows, there is described a non-limiting example of a preferred embodiment which is visualized in the accompanying drawings, in which:

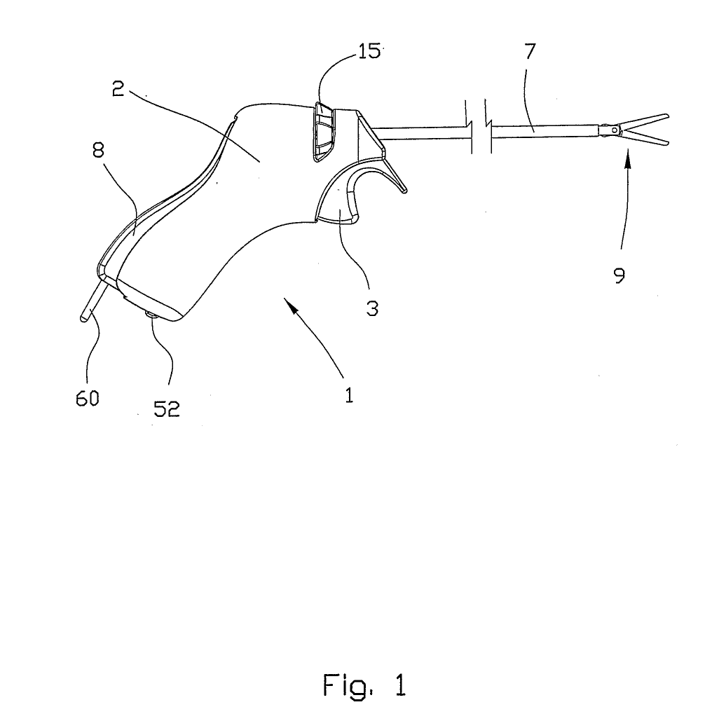

FIG. 1 shows a view of a laparoscopic instrument in accordance with the present invention, in which an actuator in the grip of the instrument is not subjected to an external force, a position which will be referred to hereinafter as non-activated, and in which an effector in the form of a pair of scissors which is placed in the cantilever end portion of a tubular element, is in an open position.

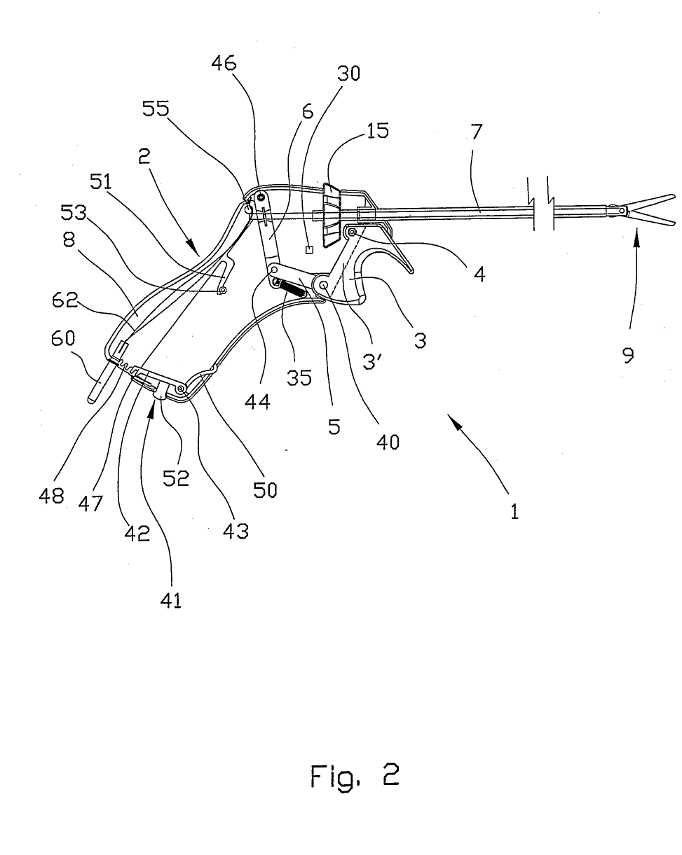

FIG. 2 shows a view of the instrument of FIG. 1, in which a cover has been removed from the grip.

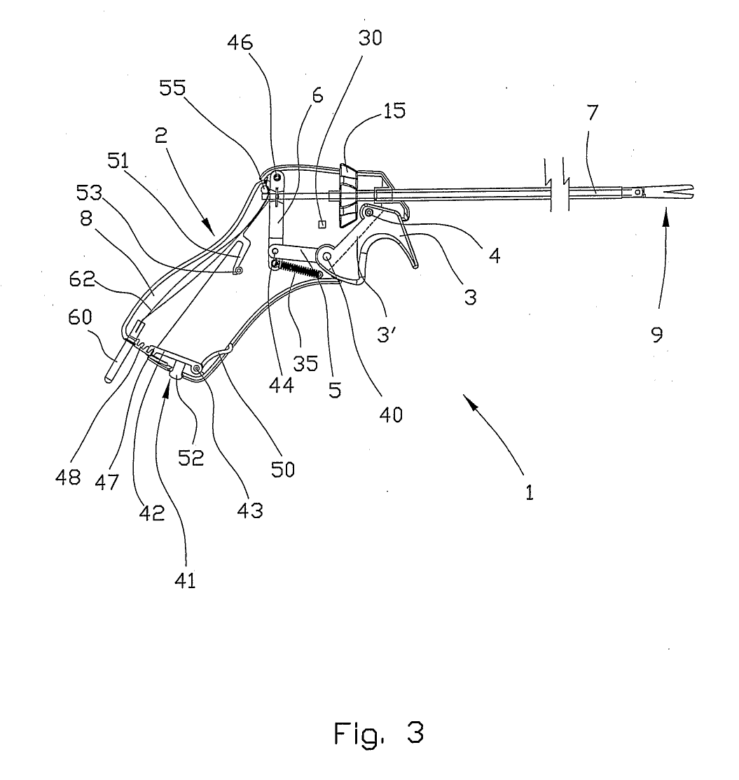

FIG. 3 shows a view of the instrument of FIG. 2, but in which the actuator has been subjected to an external force which has rotated the actuator some way into the grip and in which the scissors of the instrument are partly open.

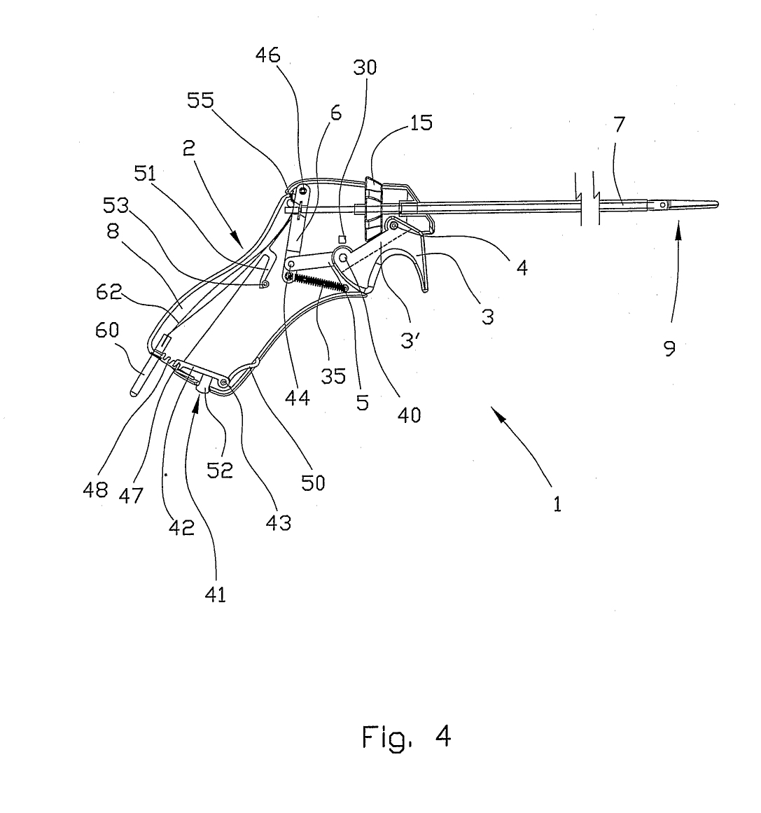

FIG. 4 shows a view of the instrument of FIG. 2, but in which the actuator has been subjected to an external force which has rotated the actuator all the way into the grip, a position referred to hereinafter as activated, and in which the scissors of the instrument are completely closed.

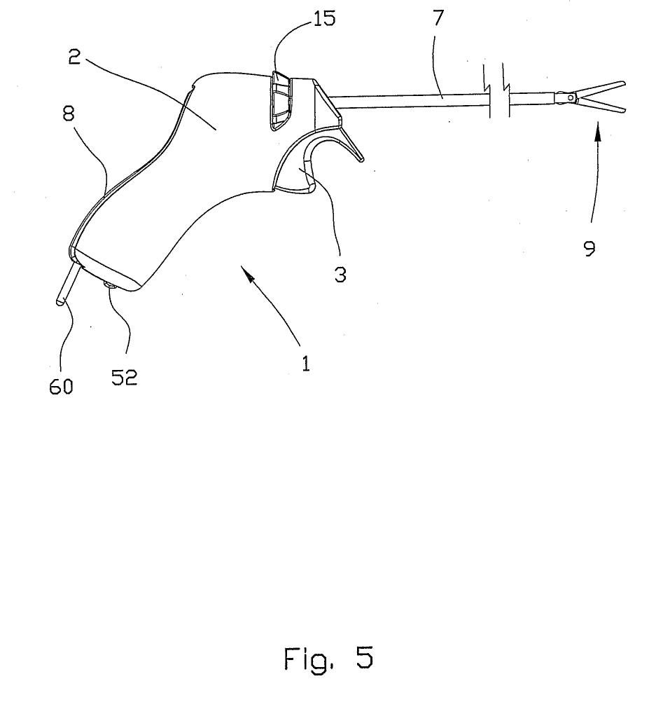

FIG. 5 shows the instrument of FIG. 1 after an adjustable back portion has been placed close to its innermost position.

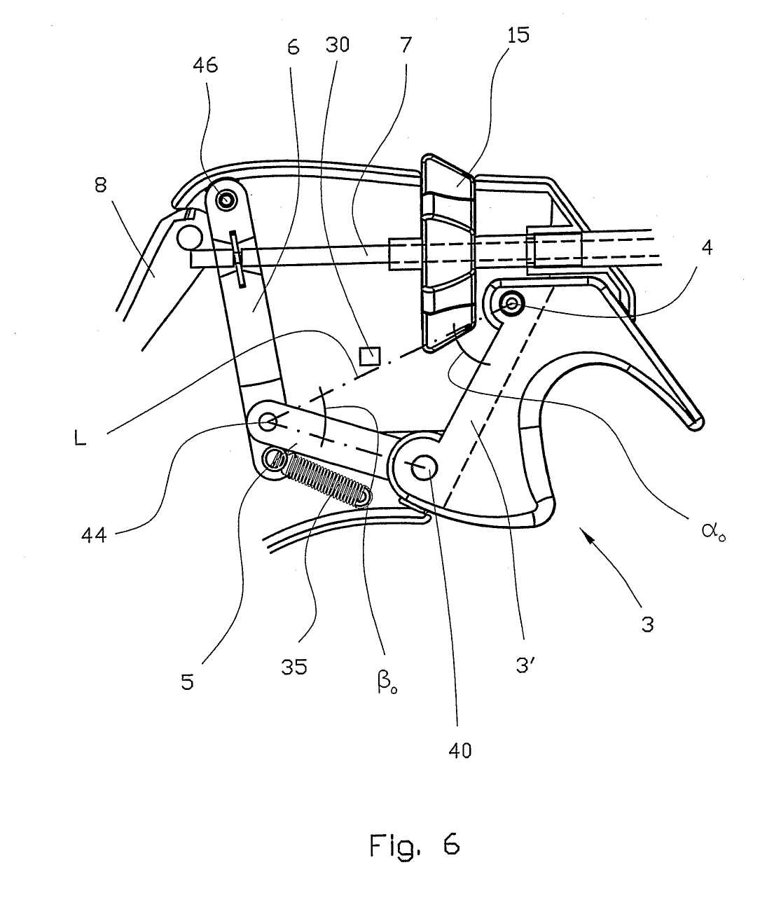

FIG. 6 shows, on a larger scale a view of the linkage of FIG. 2.

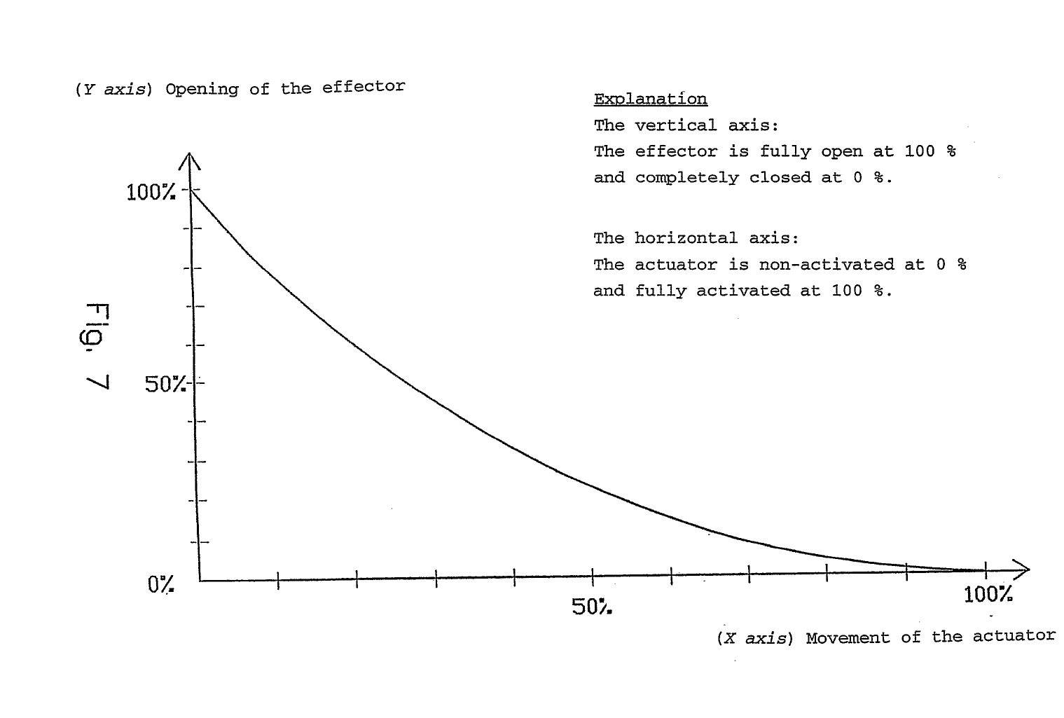

FIG. 7 shows a graphical representation of the relationship between the relative movement of the effector and the relative movement of the actuator.

In the drawings the reference numeral 1 indicates a laparoscopic instrument which is constituted by a grip 2 which is provided with a trigger or actuator 3 which is rotated, when subjected to an external force, about an axle 4 in a movement through an angle α₀, the actuator 3 being connected in a manner known in itself via a linkage 3′, 5, 6 to a tubular element 7 of a type known in itself. The tubular element 7 is placed in such a way that it projects from the grip 2. At its cantilever end portion the tubular element 7 is provided with an effector 9 in the form of a pair of scissors which can be used in laparoscopic surgery, for example.

The tubular element 7 is rotatable about its longitudinal axis. The rotation is controlled by means of a rotary wheel 15 placed in the upper portion of the grip 2, the tubular element 7 being fixedly connected to the rotary wheel 15, and, rotationally, the effector 9 being fixedly connected to the tubular element 7. By rotating the wheel 15, said tubular element 7 and the effector 9 may be rotated relative to the grip 2 in a manner known in itself.

In the FIGS. 1 and 2 the actuator 3 is unloaded, by external forces and is in its non-activated position. The actuator 3 is urged into this position by a biasing element in the form of a spring 35 connected between the linkage element 6 and a portion of the grip 2 in such a way that the biasing force is transmitted via the linkage element 5 as a compressive force on the actuator 3, causing this to be rotated about the axle 4 into its non-activated position. When the actuator 3 is non-activated, the effector 9 is in its fully open position. A person skilled in the art will appreciate that in an alternative embodiment the effector 9 may be in its fully closed position when the actuator 3 is in its non-activated position.

In an alternative embodiment (not shown), the instrument is provided without a biasing element. When an external force on the actuator 3 ceases, the actuator 3 will remain, in this alternative embodiment, substantially in the position it was in when the force ceased.

The linkage includes a link element 3′ formed by a portion of the actuator 3 (shown in a broken line in the figures) and a link element 5 connecting the link element 3′ to a link element 6. The link element 3′ of the actuator 3 can be rotated about an axle 4 connected to the grip 2. The rotation of the link element 3′ is limited between a stopping device 30 projecting from a portion of the internal surface of the grip 2, and, for example, the abutment of one end portion of the actuator 3 on a portion of the grip 2.

The link element 5 is rotatably connected at a first end portion to an end portion of the link element 3′ in a rotary connection 40, and is rotatably connected at a second end portion to a lower end portion of the link element 6 in a rotary connection 44. At its second end portion the link element 6 is rotatably connected to the grip 2 in a rotary connection 46.

The tubular element 7 of a type known in itself is connected to an upper portion of the link element 6 and activates the effector 9 in response to the pendulum movement of the link element about the rotary connection 46.

When the actuator 3 is non-activated, the link elements 3′ and 5 form an angle α₀ and an angle β₀, respectively, with the straight line L between the rotary connection 4 and the rotary connection 44, see FIG. 6.

When an external force is applied to the actuator 3, the link elements 3′ and 5 will be rotated up towards the line L until the actuator 3 is brought to bear on the mechanical stopping device 30. In this position, with the actuator fully activated, the link elements 3′ and 5 and also the rotary connection 40 will preferably coincide with the line L. In the FIGS. 3 and 4 the actuator 3 is shown in a position partially activated and near-fully activated, respectively. Correspondingly, the effector 9 is in a partially closed and in a substantially closed position, respectively. When the actuator 3 is subjected to an external force by a finger, not shown, and the angles α₀ and β₀ of the link elements 5 and 3′ are reduced to the angles α and β, respectively, the rotary connection 44 will be moved a distance ΔL. This movement ΔL causes rotation of the link element 6 about the rotary connection 46 and involves, at the same time, that the tubular element 7, whose end portion is connected to a portion of the link element 6 is moved, so that the effector 9 is moved in such a way that the jaw or opening is reduced. The movement ΔL of the rotary connection 44 from a position, in which the actuator 3 is non-activated, and until the actuator 3 is in an activated position, may somewhat simplified be expressed by the following formula:

ΔL=L_{3′}*cos α+L₅*cos β−(L_{3′}*cos α₀+L₅*cos β₀)

in which:
L₃, is the length of the link element 3′;
L₅ is the length of the link element 5;
α and β are the angles that the link elements 3′ and 5 form to the line L when the actuator 3 is in the activated position; and α₀ and β₀ are the largest angles that the link elements 3′ and 5 form to the line L when the actuator 3 is non-activated.

In FIG. 7 is shown a graphical representation of the relationship between the relative movement of the effector 9, for example the degree of the “jaw” of a gripping tool or scissors, in proportion to the relative movement of the actuator 3 in the grip 2, for one embodiment of the instrument in accordance with the present invention. The vertical axis represents the degree of jaw or opening, in which 100% is a maximum jaw and 0% is a fully closed jaw. The horizontal axis represents the movement of the actuator 3 between a non-activated position (0%) and fully activated position 100%. The representation shows that by approximately 50% activation of the actuator 3, the jaw of a tool will be approximately 75% closed, that is to say that the “transmission” between the movement of the actuator 3 and the opening of the effector 9 is relatively great in the normally “ineffective” range and relatively small in the normally effective range with the jaw of the effector 0-50% open. In the effective range low transmission ratio is very useful to the surgeon, so that the best possible control is achieved in handling vital organs, for example.

Another important property of the invention is that the effect of the force applied to the actuator 3 is substantially in inverse proportion to the “transmission” between the relative movement of the actuator 3 and the relative movement of the effector 9 or the tool jaw, which involves that the force of the effector 9 is the greatest in the normally effective range with the jaw of the effector 0-50% open.

The transmission ratio between the movement of the actuator 3 and the movement of the effector 9 has the effect that the user, for example a surgeon, experiences a more efficient instrument with improved control in the normally effective working range, which contributes to the extra energy required when using prior art laparoscopic instruments in relation to open surgery, being reduced to a considerable degree.

The instrument 1 is provided with an adjustable back portion 8 which is shown in the FIGS. 1-4 in its most projecting position. The back portion 8 is locked in the position by means of a locking member 41 which is formed by a locking element 42 which is rotatably connected at its first end portion to a portion of the grip 2 in a rotary connection 43. At its second end portion the locking element 42 is provided with a claw member 47 which is arranged to grip across one of several (four are shown) dogs 48 complementary to the claw member 47, which are placed in and project from an internal bottom portion of the back portion 8. The locking element 42 is biased by means of a spring member 50 in such a way that the claw member 47 is brought to bear across the dog 48. In a desired adjustment of the back portion 8 an adjustment button 52 is subjected to a force counteracting the force from the spring member 50, thereby disengaging the claw member 47 from the dog 48. When the claw member 47 is disengaged from the dog 48, the back portion 8 will be biased into its outermost position by means of a biasing element 51 projecting from an portion of the internal surface of the back portion 8 and bearing at its free end portion on a counter-element 53 projecting from a portion of the internal surface of the grip 2.

In the figures the back portion 8 is shown rotatably connected to the grip 2 in a rotary connection 55 placed at a top portion of the grip 2. A person skilled in the art will appreciate that the rotary connection for the adjustable back portion 8 may be positioned elsewhere, for example in the bottom portion of the grip 2, and that the adjustable back portion 6 may be provided with several rotary connections and have other configurations than those shown in the figures.

In alternative embodiments (not shown), the grip of the instrument may be provided with possibilities of adjustment also in a belly portion of the grip 2 and/or in one or both side portions.

In FIG. 5 the adjustable back portion 8 is substantially moved into the grip 2 so that the grip 2 takes its near-least projecting position and the distance between the actuator 3 and the back portion 8 will be the smallest possible. The back portion 8 of the grip 2 may be secured in a number of intermediate positions between the position shown in FIG. 5 and the position shown, for example, in FIG. 1. Such a possibility of adjustment is important in order to achieve a best possible adaptation of the grip 2 to the surgeon's hand size, so that the user comfort will be optimal.

In the figures the lower end portion of the adjustable back portion 8 is provided with a connecting device 60, known in itself, for electrical energy. The purpose of the connecting device 60 is to enable the connection of power which is carried via wires 62 to the effector 9, so that monopolar cutting can be carried out in connection with surgery. The connecting device 60 may be placed in other portions of the instrument 1 than what is shown.

A person skilled in the art will appreciate that the actuator 3, which is shown in the present exemplary embodiment with a so-called open finger-grip, may be provided with an annular or closed finger-grip.

In an alternative embodiment (not shown), the instrument is provided with a ratchet mechanism which is constituted by a ratchet bar and a substantially complementary pawl, the ratchet mechanism being arranged for selectively locking of the actuator 3 in a desired position. In a preferred embodiment the ratchet bar forms an integral part of the linkage 6. The pawl is arranged to be selectively engaged with or disengaged from the ratchet bar by means of a connection switch which is placed in a portion of the grip 2, and which is connected to the pawl via a flexible element which is arranged to exert a biasing force on the pawl. The connection switch and the actuator 3 are preferably arranged to be manipulated independently of each other.

### Text in FIG. 7

(Y axis) Opening of the effector
(X axis) Movement of the actuator

### Explanation

The vertical axis:
The effector is fully open at 100%
and completely closed at 0%.
The horizontal axis:
The actuator is non-activated at 0%
and fully activated at 100%. cm 1.-7. (canceled)

8. An instrument for use in laparoscopic surgery, comprising a grip which is provided with an actuator which is arranged to effect, via a linkage, the manipulation of an effector placed on a first end portion of a tubular element, the tubular element extending at its second end portion into the grip of the instrument and being connected to a portion of the linkage, wherein the linkage is formed by a scissors link which is connected at a first end portion to the grip by a first rotary connection, and by a lever which is connected at a second end portion to the grip in a fourth rotary connection, the scissors link being connected at its second end portion to a first end portion of the lever by a third rotary connection.

9. The instrument in accordance with claim 8, wherein the tubular element is connected to the lever in a portion between the third rotary connection and the fourth rotary connection.

10. The instrument in accordance with claim 9, wherein the instrument is provided with a biasing element arranged to bias the actuator into the most projecting position possible.

11. The instrument in accordance with claim 10, wherein the biasing element is formed by a spring.

12. The instrument in accordance with claim 9, wherein the grip is provided with at least one adjustable portion, so that the grip can be size-adjusted to different user requirements.

13. The instrument in accordance with claim 12, wherein the instrument is provided with a biasing element arranged to bias the actuator into the most projecting position possible.

14. The instrument in accordance with claim 13, wherein the biasing element is formed by a spring.

15. The instrument in accordance with claim 12, wherein one of the at least one adjustable portion is placed in a back portion of the grip.

16. The instrument in accordance with claim 15, wherein the instrument is provided with a biasing element arranged to bias the actuator into the most projecting position possible.

17. The instrument in accordance with claim 16, wherein the biasing element is formed by a spring.

18. The instrument in accordance with claim 9, wherein the tubular element is connected to the lever in a portion which is located closer to the forth rotary connection than to the third rotary connection.

19. The instrument in accordance with claim 18, wherein the instrument is provided with a biasing element arranged to bias the actuator into the most projecting position possible.

20. The instrument in accordance with claim 19, wherein the biasing element is formed by a spring.

21. The instrument in accordance with claim 18, wherein the grip is provided with at least one adjustable portion, so that the grip can be size-adjusted to different user requirements.

22. The instrument in accordance with claim 21, wherein the instrument is provided with a biasing element arranged to bias the actuator into the most projecting position possible.

23. The instrument in accordance with claim 22, wherein the biasing element is formed by a spring.

24. The instrument in accordance with claim 21, wherein one of the at least one adjustable portion is placed in a back portion of the grip.

25. The instrument in accordance with claim 24, wherein the instrument is provided with a biasing element arranged to bias the actuator into the most projecting position possible.

26. The instrument in accordance with claim 25, wherein the biasing element is formed by a spring.

27. The instrument in accordance with claim 8, wherein the grip is provided with at least one adjustable portion, so that the grip can be size-adjusted to different user requirements.

28. The instrument in accordance with claim 27, wherein the instrument is provided with a biasing element arranged to bias the actuator into the most projecting position possible.

29. The instrument in accordance with claim 28, wherein the biasing element is formed by a spring.

30. The instrument in accordance with claim 27, wherein one of the at least one adjustable portion is placed in a back portion of the grip.

31. The instrument in accordance with claim 30, wherein the instrument is provided with a biasing element arranged to bias the actuator into the most projecting position possible.

32. The instrument in accordance with claim 31, wherein the biasing element is formed by a spring.

33. The instrument in accordance with claim 8, wherein the instrument is provided with a biasing element arranged to bias the actuator into the most projecting position possible.

34. The instrument in accordance with claim 33, wherein the biasing element is formed by a spring.
